(19) United States
(12) Patent Application Publication
(10) Pub. No.: US 2019/0333648 A1
(43) Pub. Date: Oct. 31, 2019

(54) SYSTEM AND METHOD FOR BUILDING, USING AND SHARING A CORPUS OF SCIENTIFIC LITERATURE FOR A LIFE SCIENCE COMPANY

(71) Applicant: LifeScienceIT LLC, Novato, CA (US)

(72) Inventor: Yuehua Cao, Novato, CA (US)

(21) Appl. No.: 15/962,750

(22) Filed: Apr. 25, 2018

(51) Int. Cl. G16H 70/00; G06F 17/30

(52) U.S. Cl. CPC G16H 70/00; G06F 17/3056; G06F 17/30554

(57) ABSTRACT

A method and system for building, managing and using a corpus of scientific literature for a life science company. The system provides metadata and tools for interacting with, and sharing scientific literature in an efficient and compliant manner and customized to meet the unique and varied cross-functional needs of a life science company.

### BACKGROUND OF THE INVENTION

### Field of the Invention

The present invention relates to computer systems that enable a collaborative environment for building and sharing information; and more specifically, literature-based biomedical information for a life science company.

### Description of the State of the Art

The acquisition, collection and organization of scientific literature within a life science company, and the sharing of the scientific literature internally or with external collaborators is not infrequently ad hoc, disorganized and produces a system where there is a lot of duplicative and wasteful effort. This creates significant inefficiencies in the running and managing of company programs, especially those that undertake research, clinical and nonclinical studies, regulatory submissions, post market reporting and a host of other activities unique to a life science company and requiring extensive use of the same scientific literature by different persons having different skills, needs and expertise. The literature is relied on for a variety of purposes, such as supporting a hypothesis for the efficacy of a drug during a pre-clinical submission to a regulatory agency, explaining an adverse effect found during clinical trials, proposing a new program or study, or supporting a new drug application. Not only is a corpus of company literature relied on by scientists or investigators, the literature is also needed by regulatory experts who submit applications for approval and require the supporting literature as part of the submission, in a particular format and easily accessible and linked to a particular program. The diverse group of cross-functional professionals in a company need to make use of the documents in different ways, and need to communicate with each other in a collaborative way by sharing and relating literature to a program, while respecting such matters as copyrights and avoiding duplicative effort in respect to the purchasing or copying of article content. In short, an organized and highly interactive ecosystem for a life science company's literature sources and employed as a computer implemented system would be very valuable.

To meet the needs of literature management in the life science company a system is needed that organizes the content among the users of the system in a way that is customized for how the life science company operates and uses such literature. There is a special need to keep track of literature purchases, such as purchased copyrights or to avoid having different persons in the company purchase the same copyright because he/she didn't know the literature had already been purchased because previously the literature had been acquired under a different program that he/she had not been involved, a different person having different interests in the literature had purchased it but saved it locally, or the insightful aspects of the literature in formulating a hypothesis of indicating a direction of research, or content for CRF form, etc. were not made known to others who had considered using the literature.

Previous attempts in the art are not satisfactory and fall short of meeting these needs. For example RIGHTFIND by the Copyright Clearance Center offers a tool for searching through and organizing documents for general use (not specific to a particular industry). RIGHTFIND is a more generic solution for scientific and technical community. It is not made especially for a life science and, as a consequence, RIGHTFIND fails to satisfy all the special needs of a life science company in respect to literature management. For example, RIGHTFIND does not provide a document management system that automatically allows a user to associate a document as ready for use with a regulatory submission or related to a program at the time of purchase, allows sharing literature readily with external collaborator(s) in a way compliant with the Sunshine Act, or provides access to a special library where only invited users who accept an invitation will be able to access the special library. This part is important for a business development group within the company working on a highly confidential new program. The system of document management for the life science company needs to be flexible in this regard, allowing for efficient sharing and acquiring of literature while providing certain safeguards against the release of information discussed when literature is being shared amongst employees.

Government agencies offer tools and working environments for life science research and provide as part of this environment access to a database of literature. One example is PUBMED, which is a free search engine accessing primarily the MEDLINE database of references and abstracts on life sciences and biomedical topics. The United States National Library of Medicine (NLM) at the National Institutes of Health (NIH) maintains the database as part of the Entrez system of information retrieval. https://www.nlm.nih.gov/pubs/factsheets/pubmed.html. The Entrez Programming Utilities (E-utilities) are a set of nine server-side programs that provide a stable interface into the Entrez query and database system at the National Center for Biotechnology Information (NCBI). The E-utilities use a fixed URL syntax that translates a standard set of input parameters into the values necessary for various NCBI software components to search for and retrieve literature, e.g., using the PubMed search engine. The E-utilities are therefore the structured interface to the Entrez system, which currently includes 38 databases covering a variety of biomedical data, including nucleotide and protein sequences, gene records, three-dimensional molecular structures, and the biomedical literature. https://www.ncbi.nlm.nih.gov/books/NBK25501/

A life science company that makes use of these agency tools and database through a government sponsored site, e.g., by constructing a software system within the company that provides basic download, file structures and memory for purchasing and using literature, and keeping track of such literature using, e.g., a spreadsheet, often runs into problems of waste or unnecessary expense and disorganization or many hours spent updating and keeping track of literature, formatting literature and sharing of literature among cross-functional groups within the company. When a life science company relies on a simple, computer-implemented library of literature to manage their corpus of scientific literature, the following things can happen. First, there is the problem of duplicate orders. If a company has a central repository for all ordered literature, a user needs to visually check all full-text literatures to see if it's already there before ordering an article; if he checks wrong or someone ordered but forgot to put it there or if a company does not have a central repository, duplicate orders will surely be created and money wasted. Second, a library system constructed as a general purpose tool for organizing literature does not have the linking between a document and its metadata, e.g., company program information-type linked to metadata. Third, most of the literature received is not at the grade of submission-ready; i.e., copies of the literature print are not sufficiently clear and legible, have spots, marks, etc. Ideally the system should allow the addition of a new version on top of existing version and record who added the additional version(s) at what time. There is also the problem of keeping track of expenses, especially in light of documents purchased or shared with health care professionals (HCP). Gifts to health care professionals, e.g., physicians, above $10 in value must be reported under the Sunshine Act. See 42 CFR Part 403. Similar reporting requirements exist in Europe.

Another limitation in the art relating to managing a corpus of literature for a company's programs, is the use or proliferation of acronyms that often evolve during the course of a program. Many scientific or regulatory concepts are often used in the context of communication without any definition of the acronym being given. Resources exist on the internet for locating definitions for acronyms. In the life sciences the FDA has a downloadable file of acronyms. However, such services are limited and fall short of a company's need to manage its use and communication of acronyms internally.

In view of the foregoing, there is a need for a system for building, managing and sharing a corpus of scientific literature that addresses one or more of the foregoing needs of a life science company, in support of the various cross-functional groups and programs priorities in an efficient and organized manner.

### SUMMARY OF THE INVENTION

According to a preferred embodiment of invention, a biodigital library (BDL) is provided that addresses the foregoing needs of a life science company. More generally, a computer-implemented method and system is provided that enables a collaborative environment for building and sharing of literature-based information for a life science company. A corpus of literature is added to, shared and linked to company programs within a computer-implemented system that accounts for the different uses of the same literature across-different groups and programs, manages effectively the purchase and re-purchase of rights and tracks how a document is used and its significance to a particular program.

The invention offers at least the following advantages:
Duplicative downloads are avoided automatically. All ordered literature, all subscribed literature, free literature, literature ordered but not yet contributed are all clearly marked in a search result. This information is recorded and tracked.
Metadata is available for any stored literature is conveniently available. This metadata includes both the bibliographic data and metadata that is company specific—e.g., literature flagged as Key literature for a program, literature that is submission-ready, or included in a writing library for a small group of individuals tasked on a specific project, what copyrights are purchased, and whether the document can be freely shared.
A link is available for each literature, or all literature in a program and can be automatically included in any email or document. And the link never changes. In contrast, for some existing systems, including those with a file share feature, if the location in memory of literature, or a folder name/location for the literature moves or changes, the link used by the program accessing the literature expires. The application portion of the system according to one aspect of invention takes account of these changes so that the link never expires, in combination with the other inventive features.
Ordering, copyright purchase and order history also linked as part of document metadata.

Compliant to copyright and regulatory requirements (e.g., Sunshine Act) A life science company makes use of literature for a wide variety of reasons. Among these are citation of literature to different regulatory agencies requiring different citation formats (e.g., EU verses US) or citation styles required by certain publications. When literature is used in several different contexts it can be extremely time-consuming formatting the citation style for compliance for a particular use. According to another aspect, there is a user interface provided that allows individuals to instantly generate citations to literature using the metadata linked to documents. This tool furthermore allows an individual to create a customized citation using the metadata. The customized citation is created instantly by simply choosing it and selecting the document to which the citation applies.

According to another aspect, there is a tool that allows a used to search for acronym definitions and make customized acronyms for internal use. Whenever a question arises as to what an acronym means—e.g., acronyms originating within a working group, when discussion or findings communicated to larger group of persons on a project, the dialogue is hard to follow due to use of unknown acronyms—the system provides a ready-accessible list of acronyms that can help further understanding of a working groups internal communications. More generally, In life science industry, there are significant numbers of acronyms and used extensively in communications, presentations, and documentations. One acronym that is well known in one function may be completely unfamiliar to people in another function; moreover, one acronym could easily resolve to multiple phrases of different meaning. The invention provides a classification system that classifies all acronyms into industry standard acronyms and custom-specific acronyms. Acronyms having different meanings may co-exist in the system according to the invention, even though they have the same acronyms; and safeguards are in place to ensure that industry standard acronyms can't be changed while custom ones can be added/edited (including indication of who changed it to enforce accountability/verify its accuracy).

The system and method of invention moreover provides literature search, acquisition, automatic metadata population, versioning, to different ways of consumptions of the literature, for users from several cross-functional groups within a life science company (Research, Clinical, Nonclinical, Medical Writing, Regulatory, Medical Affairs, Business Development, Commercial, Biometrics, Program Management, etc.).

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### BRIEF DESCRIPTION OF THE DRAWINGS

FIG. 1 is a schematic showing a system for managing, updating and using a corpus of scientific literature for a life science company.

FIG. 2 is a schematic showing the types of data and information maintained on a database of the system of FIG. 1

FIG. 3A is a screenshot displayed to a user of the system of FIG. 1 and showing search results for literature searched using the system.

FIG. 3B is a screenshot showing metadata associated with literature acquired/residing on the database.

FIG. 3C, 3D are screenshot/schematic showing an order history and literature sharing features of the system of FIG. 1.

FIG. 4A is a flow diagram describing computer-implemented steps in response to user input for acquiring literature from an external database.

FIG. 4B is a flow diagram describing computer-implemented steps for purchasing rights in literature.

FIG. 4C is a flow diagram describing computer-implemented steps in response to user input for sharing literature from the database when said literature is, or is not subject to a copyright fee and Sunshine Act/Spend Transparency Regulations.

FIG. 5 is a flow diagram describing computer-implemented steps in response to user input for searching for acronyms or creating/editing acronym definitions.

FIG. 6A is a screenshot/schematic showing citation styles and a pop-up window for creating custom citation styles.

FIG. 6B is a flow diagram describing computer-implemented steps in response to user input for either creating a new citation style or applying a citation style in written works.

### DETAILED DESCRIPTION

In the description like reference numbers appearing in the drawings and description designate corresponding or like elements among the different views.

For purposes of this disclosure, the following terms and definitions apply:

Whenever reference is made to a “company” it means a life science company that provides health related products and services, e.g., drugs or medical devices, that are subject to approval by a regulatory agency, e.g., the FDA.

As used herein “literature” means one or more articles from scientific journals, books, chapters, posters, conference proceedings, thesis, etc. including academic journals and covering medicine, nursing, pharmacy, dentistry, veterinary medicine, and/or health care. Full-text literature means the entire literature, publication, article, thesis, research report, etc., as opposed to only an Abstract.

PUBMED is a free search engine accessing primarily the MEDLINE database of references and abstracts on life sciences and biomedical topics. The United States National Library of Medicine (NLM) at the National Institutes of Health (NIH) maintains the database as part of the Entrez system of information retrieval. https://www.nlm.nih.gov/pubs/factsheets/pubmed.html

The Entrez Programming Utilities (E-utilities) are a set of nine server-side programs that provide a stable interface into the Entrez query and database system at the National Center for Biotechnology Information (NCBI). The E-utilities use a fixed URL syntax that translates a standard set of input parameters into the values necessary for various NCBI software components to search for and retrieve literature, e.g., using the PubMed search engine. The E-utilities are therefore the structured interface to the Entrez system, which currently includes 38 databases covering a variety of biomedical data, including nucleotide and protein sequences, gene records, three-dimensional molecular structures, and the biomedical literature. https://www.ncbi.nlm.nih.gov/books/NBK25501/

As used herein, a “user” of the computer system according to the invention is a person associated with the life science company, such as a researcher, programmer, regulatory specialist, biostatistician, scientist, Medical doctor, etc., given access rights to a computer implemented system according to the invention. The user accessing the system has a function within the company such as Research, Clinical, Nonclinical, Medical Writing, Regulatory, Medical Affairs, Business Development, Commercial, Biometrics, Program Management, etc. The system is accessed by the user using a user interface, e.g., a monitor or display, whereby the user can access contents of a database part of the system, make changes, upload literature, etc. using an input device.

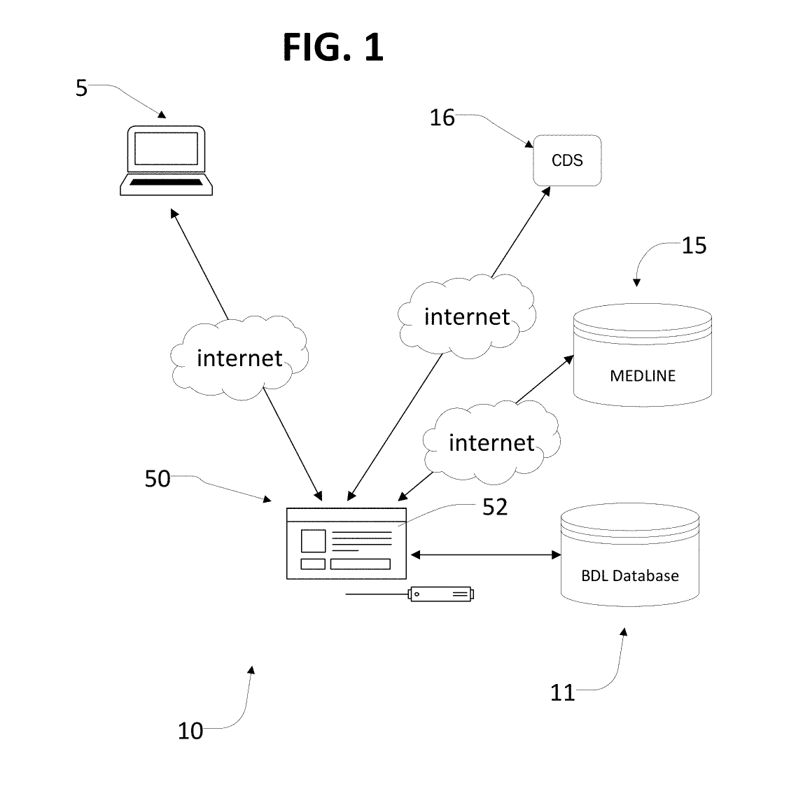

FIG. 1 shows an example of a computer-implemented system 10 (e.g. the BDL system) that enables a collaborative environment for building and sharing a corpus of scientific literature for a life science company. A user 5 residing at a laptop or desktop computer or a mobile device accesses and/or participates in this collaborative environment via a server application 50, which may be accessed via a web browser through a local area network (LAN) wired or wireless, or over the internet.

The application 50 is a web-based application where access to the application is provided through a web browser, or mobile application, where the app is specifically designed for mobile devices. The application 50 includes machine-executable instructions in the form of software that when executed by a processor provides various forms of functionality, which may include generating a GUI, read and write of metadata and files to/from a database 11, communicating with a remote server, managing various APIs, etc. as part of system 10.

Application 50 presents the user 5 with a workflow space including graphical user interface (GUI) 52, which has a series of drop-down menus used to access/organize information, input information into the system, initiate queries, search online or commence purchases of literature, share information, format data, access/modify user rights, etc. as described in greater detail herein.

The application 50 is connected to memory accessed through a database server for storing data and information related to the corpus of scientific literature. The database 11 provides read and write access to non-volatile memory.

Application 50 also provides the user with access to an external database 15 of literature accessible over a network, such as the internet. Literature in the database 15 may be searched by, e.g., keywords, category, author, publication data, etc. using a search engine provided for accessing the external database 15. The search engine may be accessed directly from the GUI 52 using an application programming interface (API) to the database search engine, e.g., an API provided by E-Utilities available from the NCBI. In the preferred embodiment the external database 15 is the MEDLINE database of literature and the search engine is PUBMED. The system may also use a content delivery service 16 (CDS 16) to facilitate purchase and transfer of literature found on database 15 to the user 5, e.g., via email enclosing a link to the literature on a file server over the internet. The user 5 then uploads the literature to the database 11, as described in greater details below. The CDS 16 may represent a third party service that maintains an account of literature purchased by the life science company.

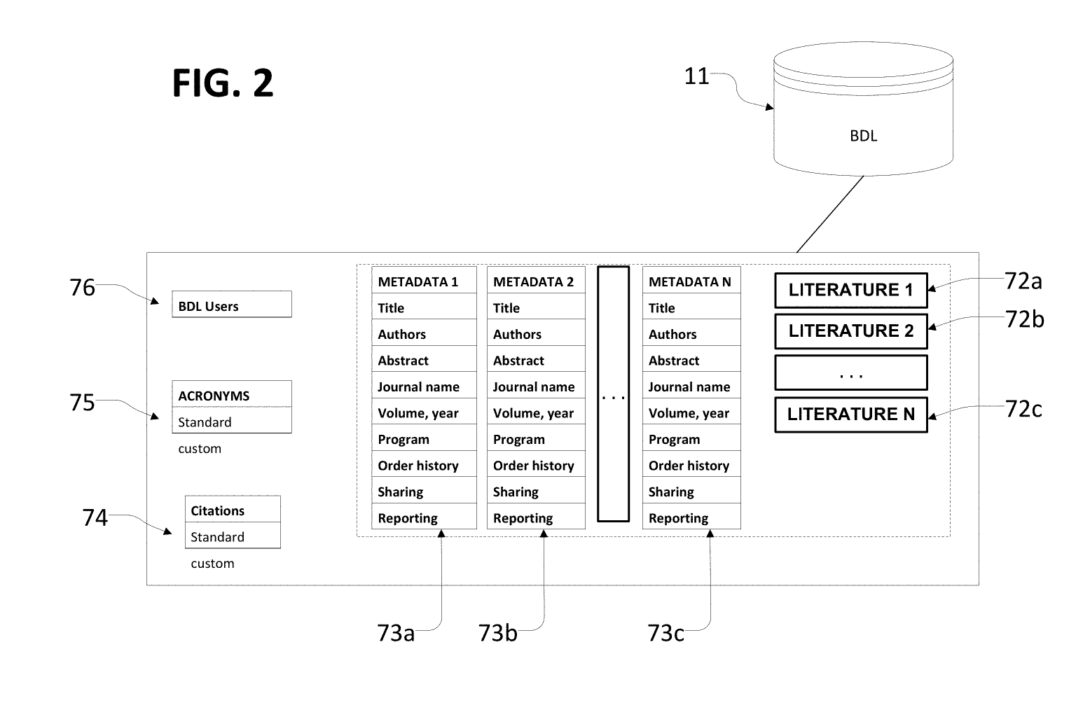

FIG. 2 shows a schematic indicating the contents of the database 11, which in the preferred embodiment is a database owned and operated by, or on behalf of the company. Shown is the type of information stored and accessible from the database (e.g. SQL database) using software resources/utilities for interrogating the database, reading and writing from the database, etc. Shown in schematic form in FIG. 2, is a description of the information in general that resides in the database. Each of the information/data categories are described in more detail below. The arrangement of boxes in FIG. 2 is not meant in any way to imply a particular formatting or data structure for information or data contained in the database 11, unless specifically noted below.

The information stored in database 11 includes the corpus of literature (“literature 1,” “literature 2” . . . “Literature N”). Each of the literature 72a, 72b, 72c is associated or linked to the metadata 73a, 73b, 73c recorded for each literature (“metadata 1”, “metadata 2” . . . “metadata N”). The information associated with each of the metadata is described in greater detail below. By associated or linked to the literature means that whenever an inquiry or search of the literature is made, the metadata is made available to the one requesting or using the literature, thereby providing an extensive amount of structured data about the literature specific to the needs of a life science company. The depiction of all this information within a single record in FIG. 2 does not imply that all this information is kept in one place in memory. The schematic is made this way only to facilitate a greater understanding of the kinds of information that is saved and associated with each of the literature stored in the database 11.

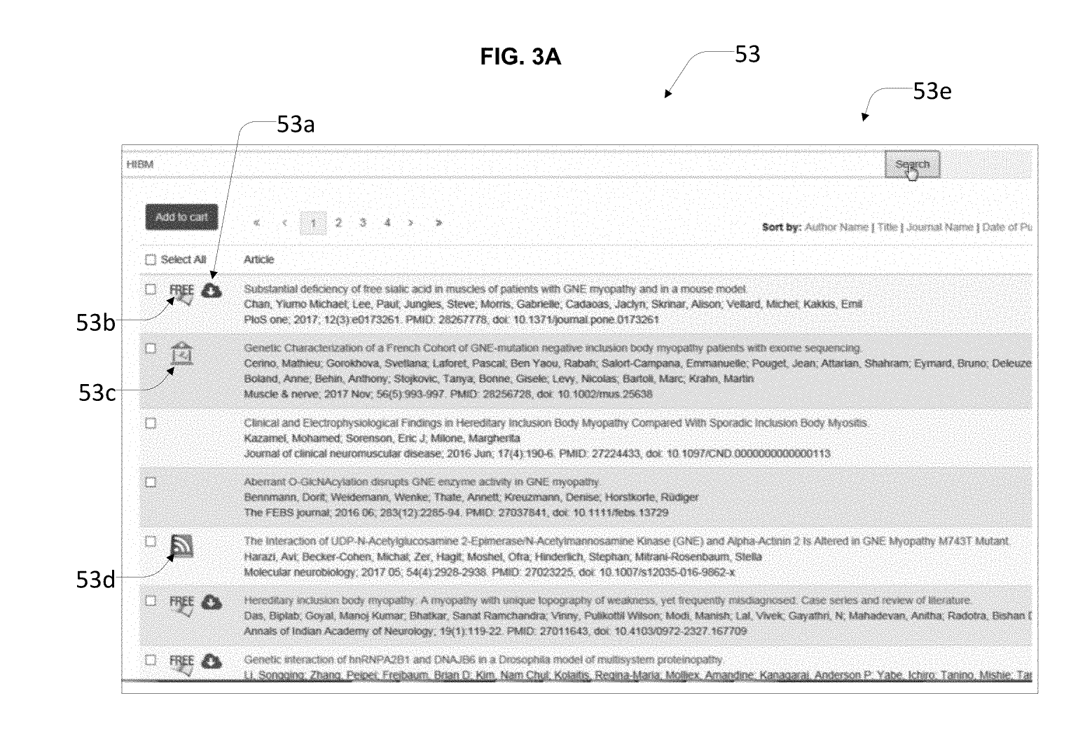

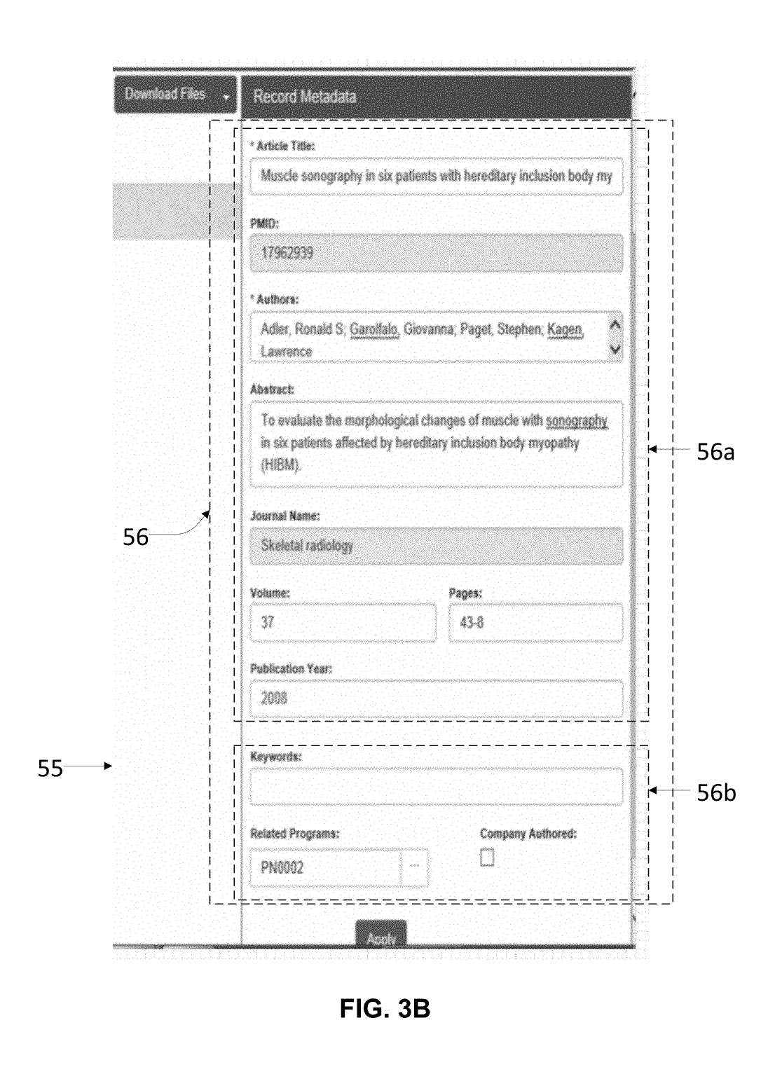

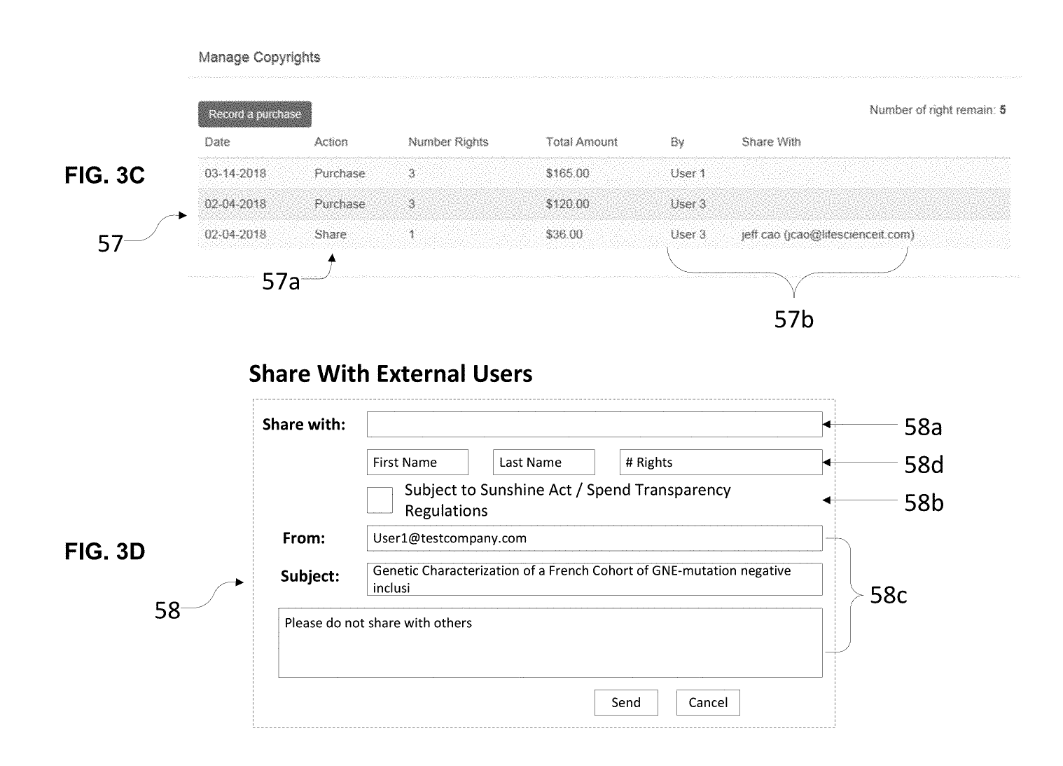

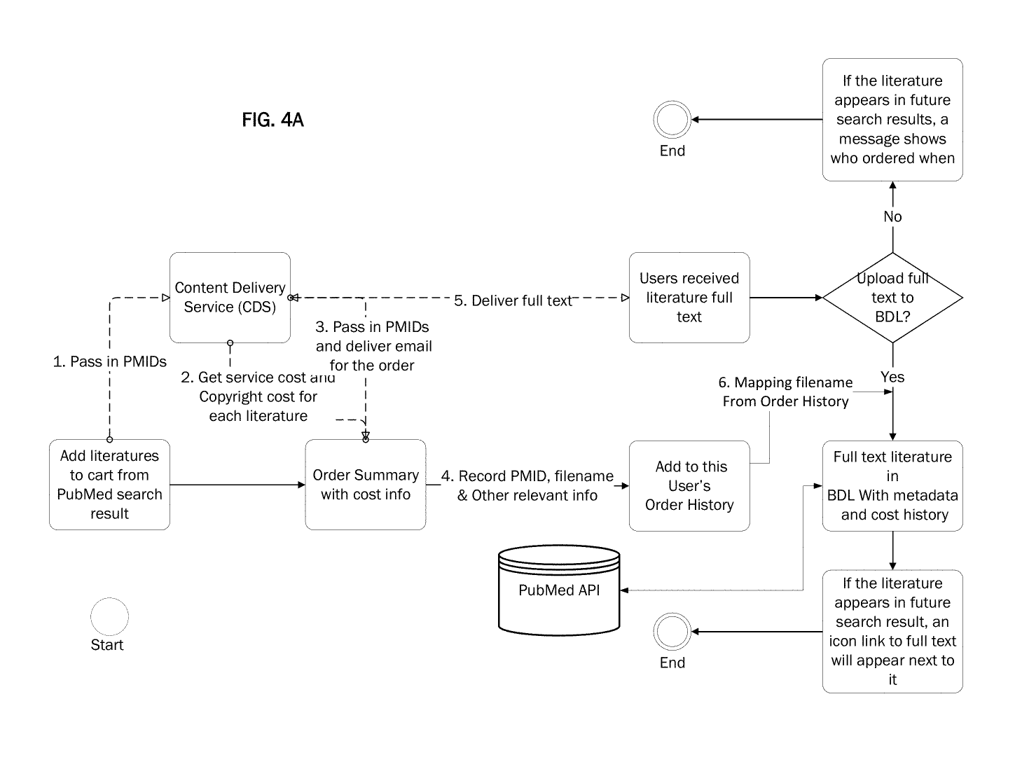

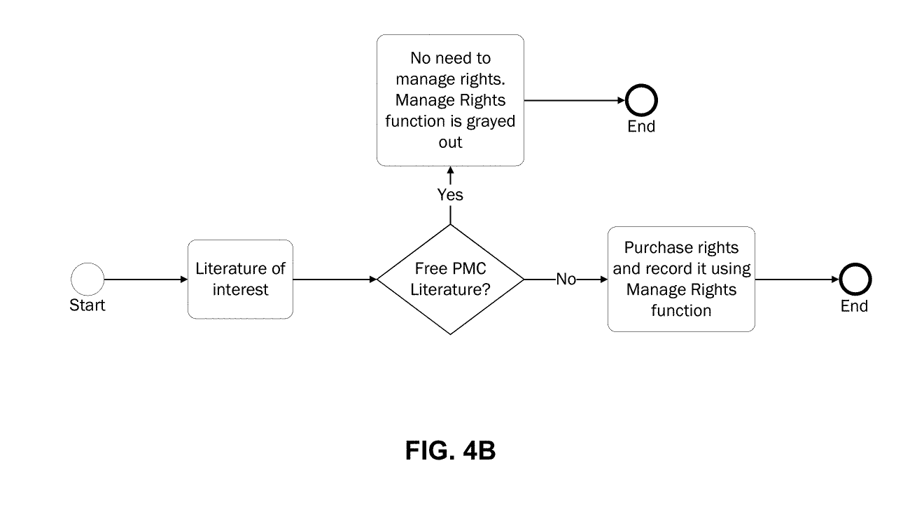

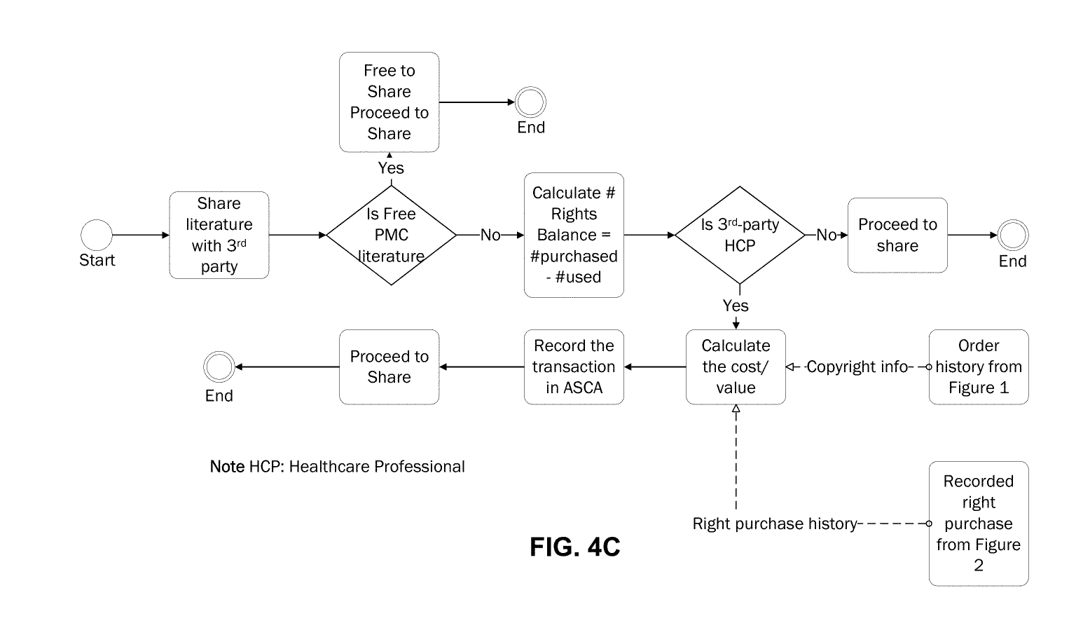

The metadata for literature 73a, 73b, 73c includes the bibliographic data (e.g., title, author, abstract, journal etc.), the program or user specific data (program number, comments, importance, status—FIG. 3B) and the information associated with copyrights and sharing of the literature (FIGS. 3C, 3D, and 4A-4C).

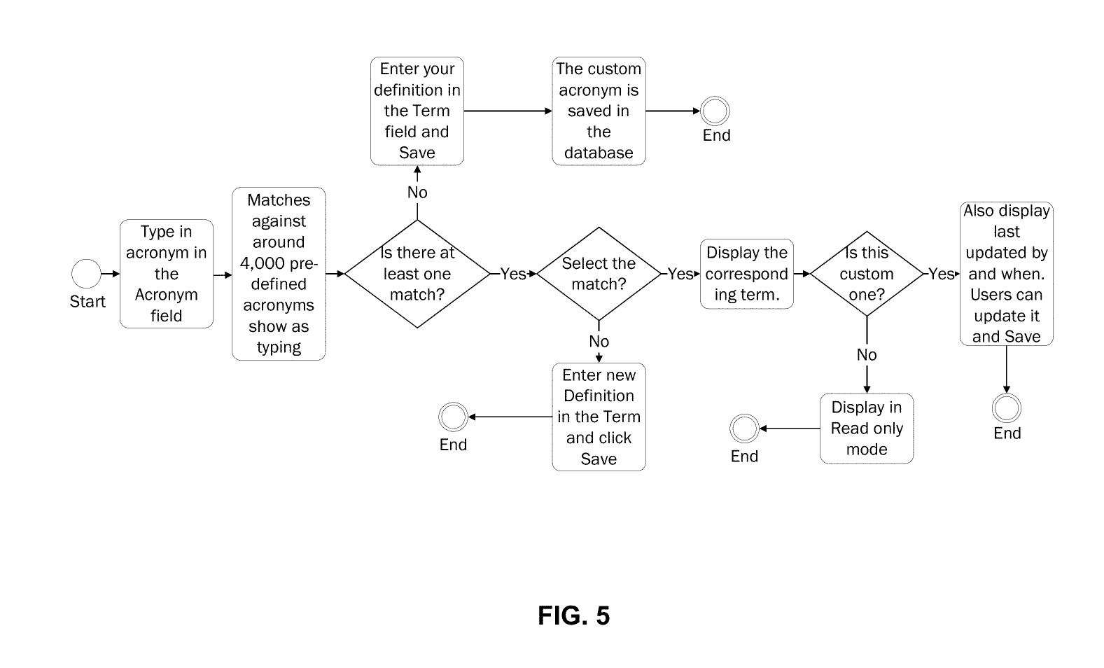

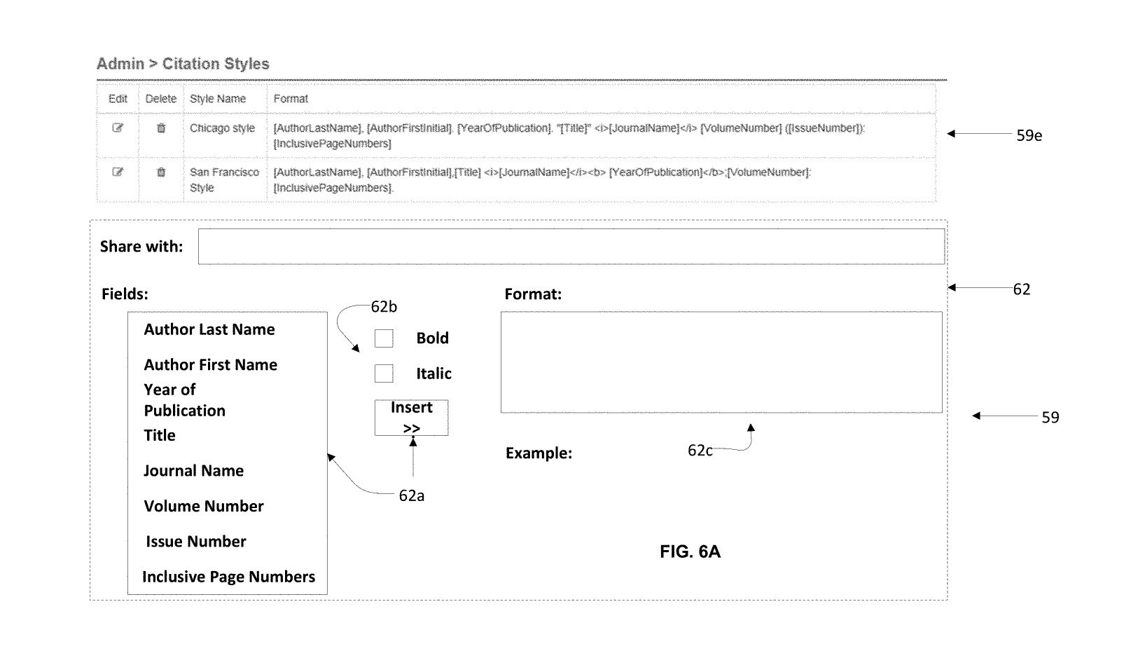

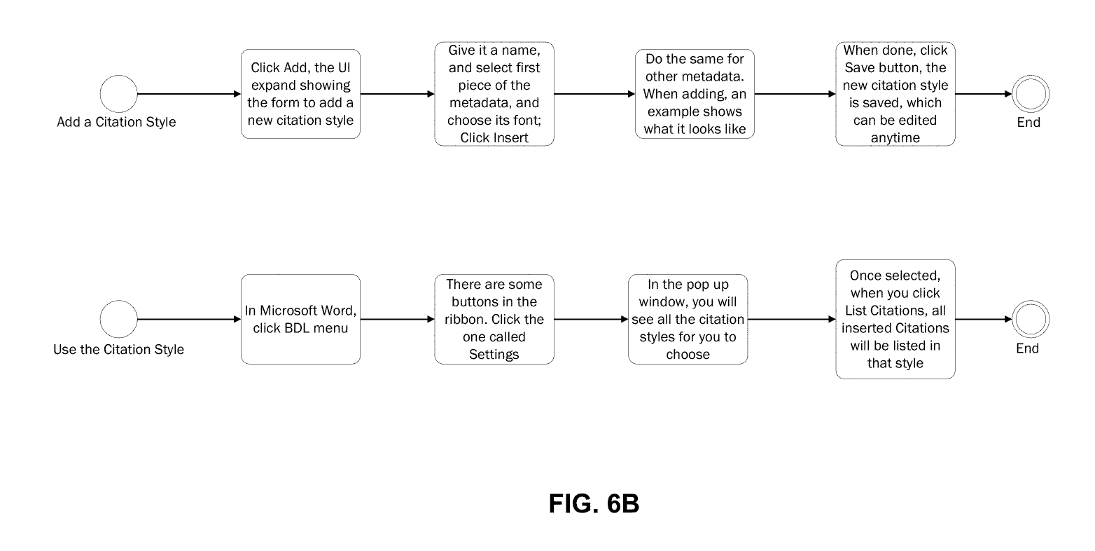

Also contained in the database are the users with associated access rights recorded, and information about the users 76, the acronym records 75 (custom and industry-stand, along with metadata about the acronyms such as users contributing to or adding to the acronyms list—FIG. 5), and the custom and standard citations records (FIGS. 6A, 6B).

Now described are features of system 10 for searching and acquiring literature from database 15. Using GUI 52 a user searches for literature and receives in response a listing of the documents that meet the search criterion. FIG. 3A is a screenshot 53 of the display presented to the user by the GUI showing search results retrieved from the database 15, in this case results provided using PUBMED to search the MEDLINE database. The screenshot 53 shows a search function 53e for searching the external database 15 directly from application 50. The page shown provides the user with information about each of the literature returned in response to the search entered using window and search button 53e.

For each literature there is a box provided for selecting the cited literature, e.g., for download or purchase. The downloaded search results provide immediate access to the abstract, title and authors for viewing. Also shown are icons indicating different levels of availability for the literature. The icons appearing next to some of the literature shown in FIG. 3A indicate the literature is free and may be downloaded without a copyright fee (icon 53b and 53a, respectively), the literature has been previously downloaded to database 11 and is available from the system 10 (icon 53c), or the literature is available through a subscription service, to which the life science company subscribes (icon 53d). When there is no icon next to the literature description (e.g., the third and fourth listed literature) then the full text literature requires payment of a copyright fee (only the Abstract is free for viewing).

Referring to FIG. 3B, after literature is selected for downloaded (as described below in connection with FIG. 4A), the user is presented with a screenshot 55 of the display presented to the user by the GUI. This screenshot from the GUI 52 presents the user with a record metadata button that produces box 56 (as shown) that has a first portion 56a of the literature bibliographic information automatically populated by the application 50 (as described below in connection with FIG. 4A) and a second portion 56b containing information that relates the literature to one or more programs at the life science company. The first portion 56a provides in a record for the article text of the Abstract, Title, author(s) names, journal name, article ID (e.g., PUBMED ID), volume, pages within the journal, volume, etc. The portion 56a, when auto-populated with the literature information, stores this metadata in the database 11 and associates it with a copy of the literature, also saved in database 11. By storing the information in this manner the application 50 can provide various functionalities associated with the literature, as described in greater detail below.

Portion 56b of the metadata box provides fill-in/selection boxes for associating company-specific information with the literature, which is saved as metadata with the metadata extracted from the literature. The company-specific information may include, for example, a program name (e.g., PN0002) associated with the literature, a box indicating whether the literature has at least one author from the company, keywords specific to a company function, activity or area of interest, the relates to a subject discussed in the article (e.g., a key finding as summarized in the abstract does or does not support a hypothesis derived from a milestone of a clinical study under program PN0002).

In addition to the company-specific information shown in portion 56b, there is one or more of additional metadata (selectable as a tag for literature using the GUI): Key literature (literature marked as key or high relevance to a program objectives, findings, adverse indications, etc.); Submission-ready (the margin sizes of the literature document, if the bookmark of sections in the literature exist, and if there is any line/spot introduced from copying or scanning, etc. must satisfy agency's guidance); and Included/Favorite.

Related to the submission-ready metadata, the metadata linked to literature also includes a versions metadata. For example, literature first acquired is later determined as needed for a submission, but is not submission-ready. When it is determined the literature is not submission ready, then a second version is acquired. When the submission-ready version is available, the metadata is updated. This information is linked to keep track of the same literature acquired later, but for the purpose of obtaining a cleaner version of it (also tracked is user adding version at what time). Hence the system 10/application 50 can distinguish between a submission-ready and not submission ready version of the literature based on metadata, and link both copies.

Referring to FIG. 4A, there is shown a flow diagram describing a process for acquisition of, and adding literature to the database 15, and acquiring the bibliographic metadata (populated boxes of first portion 56a). The lines and arrows indicate flow direction and communication among the system 10 (via application 50), user 5, a content delivery service (CDS) and external database 15 (through the API). The CDS and database 15 are accessed over a suitable internet connection. Additional steps not explicitly shown include e-mail communication where the user (residing at client computer 5 or elsewhere) receives literature access via e-mail from the CDS.

The process starts with selecting literature for download using the GUI 52. As just explained there is an indication in the search results of whether the literature is provided for free or subject to a copyright requiring a purchase. If the literature is available for free, the user may simply click on the icon 53a or 53d to gain access to the full text. If a purchase of a copyright is needed (no icon), then the literature is added to a checkout cart accessible from GUI via screenshot 53.

In the preferred embodiment the literature is located in the MEDLINE database. For each article selected and in the cart, an identification number for the literature (PMID) (STEP 1.) is sent or passed to a content delivery service (CDS) that provides full-text copies of literature. In this example, requests are made for literature found on MEDLINE where each literature is identified with a PMID.

In response the CDS provides a service cost and a copyright cost (if one applies) for each literature selected for download. The application 50 retrieves this cost information from the CDS for each selected literature (STEP 2.) and displays it to the user via the GUI 52, which presents to the user an order summary with the cost information for each literature (not shown). Next, for the literature that the user wishes to download, it selects those literature using the GUI 52, clicks send and the application 50 sends back the corresponding PMIDs to the CDS (STEP 3.). At this point the system 10 records the PMIDs, service cost, copyright cost, filename, program number (e.g., program PN0002, which metadata is entered by the user at the time of purchase so that the purchase information can be charged to a specific company program. The other metadata described in portion 56b is entered after the literature is downloaded to database 11), user name and date stamp associated with the purchase (STEP 4.). This information is added to the user's purchase history, which is stored on database 11.

The CDS in response to the PMIDs (STEP 3.) initiates the transaction and if approved sends an e-mail communication to the user 5 for each one of the literature selected. The e-mail contains a URL for accessing a file server for download of the full text version of the literature from the database 15. The user then, using the URL, accesses the full text version of the literature and downloads it locally to his or her computer as a file (Step 5.). The user then uploads this file to the system 10 where it is stored on database 11. The recorded metadata associated with the purchase of the literature (Step 4.) is then mapped to the literature file (STEP 6.); or the metadata is associated with the file, as explained more during the discussion that makes reference to FIG. 2. The PMID for each literature is then passed to the API used to access the database, e.g., PUBMED API (STEP 7.) This API in response provides metadata in structured format that application 50 then uses to auto-populate the bibliographic metadata described earlier in connection FIG. 3B (portion 56a of metadata record).

Scientists, regulatory, etc. and the other professionals within a life science company will frequently need to review and share literature on database 11, e.g., for purposes of sharing the literature with colleagues both inside and outside the company and when a submission is made to a regulatory agency. Another aspect of System 10 is a tool that makes sharing of literature subject to copyright fees manageable and far easier to organize and keep track of than what currently exists.

The application 50 includes functionality to notify the user when literature may be shared without limitation, or shared subject to the scope of the copyright purchased for literature. Referring to FIGS. 3C and 3D, there is shown screenshots 57 and 58, respectively, of the displays presented to the user by the GUI. Screenshot 57 shows the user an order/purchase history for the rights of a piece of literature. Shown are actions 57a taken that resulted in a charge to the company account—i.e., purchase of the literature or sharing of it, the latter of which resulted in fee because the literature sharing triggers copyright license fee for this literature (the system automatically records this information as part of the process described in connection with FIG. 4A. Also shown are the persons that took this action resulting in the fees, and to whom the literature was shared 57b. Additionally shown are the number of copyrights purchased. This information is needed when deciding whether or to whom the document may be shared without requiring additional purchase, or to keep track of what has already been purchased.

Screenshot 58 is a display presented to the user by the GUI showing buttons and input windows for sharing literature outside of the company. The screenshot shows a window 58a for entering an e-mail address or user name and immediately below buttons/window 58d indicating the number of rights available or remaining after the literature is shared and the individual to whom the share is made (assuming a copyright fee applies). Also shown is a selection box 58b indicating whether the sharing is made to a health care professional, for purposes of reporting said information as required under the Sunshine Act. Without this feature available, the record keeping needed for ensuring compliance with the Sunshine Act for any literature subject to a copyright fee, especially in a large corporation, is significant. The invention provides this recordkeeping function automatically whenever any literature is shared outside the corporation. Finally, the screenshot 58 shows windows for entering the user name who is sharing the literature, the subject and text field for the e-mail 58c.

FIGS. 4B and 4C are flow diagrams describing the software functionality for managing copyrights and sharing as just discussed in connection with screenshots 57 and 58, respectively. Referring to FIG. 4B, after selecting the literature of interest the software checks whether the literature may be freely shared or subject to a copyright fee. If the former no need to manage rights and the rights part of the GUI display (portion 58d of screenshot 58) is disclosed. If the later (fee applies, then the software completes the transaction purchasing the additional right(s) and records it in the ordering history (screenshot 57).

Referring to FIG. 4C, when the user shares literature outside of the company (i.e., a 3rd party person/entity outside the scope of the purchased copyright) the flow proceeds with calculating the rights balance—i.e., purchased rights share available in account, or more purchasing needed? Then, it is checked whether the 3rd party is a health care professional (HCP), which requires reporting the share as a gift under the Sunshine Act (if not a health care professional the account charged and the literature is shared). If the 3rd party is a HCP (i.e. box 58d was checked) the software next calculates the gift value of the share (using the information from the record providing order history information) and records this gift value in the reporting record ASCA (Section 6002 of the Affordable Care Act reporting requirements). After the gift value and recipient of the gift is recorded and saved in database 11 the literature is shared with the 3^{rd} party HCP.

According to another aspect of invention, the application 50 GUI provides the user with a menu with input fields to define a special library of selected literature from the database 11 and ability to assign access rights to this library. For instance, the application 50 allows, e.g., the head of a business development group, to select a plurality of users that will have access to the library, but no one else in the company. The selected group then shares confidential business development information using the system tools and resources available, such as metadata fields linked to documents, for planning and discussion purposes. This separate library with restricted access is provided by system 10 without the need to create duplicate copies of literature. Hence, the system both organizes and keeps track of a literature without duplication or wastes and moreover allows users to discuss sensitive matters by defining working groups having access to a restricted set of literature maintained in the database.

In the life science industry there are significant numbers of acronyms and they are used extensively in communications, presentations, and documentations. One acronym that is well known in one function may be completely unfamiliar to people in another function. To make matters worse, one acronym could be resolved by different people into multiple phrases of different meaning. According to another aspect of System 10 there is a classification system available for all acronyms—both the industry standard acronyms and company or individual specific acronyms. To the extent there is overlap or conflict (industry standard vs. customized meanings) the system includes software functionality to recognize the conflicting meanings and makes both meanings available to users. Moreover the acronym system includes safeguards to ensure that industry standard acronyms cannot be changed, while custom ones (or ones used in industry but not having any one accepted meaning) can be added/edited. For these custom acronyms the GUI presents to the user a display showing who added the acronym, or who changed it.

Referring to the flow diagram of FIG. 5, the user is presented with a display for looking up or adding acronyms to the system of acronyms in the system. The flow may begin by the user typing in an acronym in a GUI input field and searching for matching acronyms. If there is no a match (nothing returned from search query in database of acronyms), the user is presented with the option to enter a definition for the acronym. This custom acronym is saved in database 11 and made accessible to others. Additionally, for this custom acronym the user who made this entry is recorded.

If the search returns one or more acronyms matching the search criteria, then the user either selects one of the matches or not. If the latter, the user enters a new definition for the same (or similar) acronym as currently exists on the system (unless an industry standard, in which case this action is not allowed) and as before the user name is recorded in the database with the new definition of a same or similar acronym to another in the system.

If the user selects a preexisting acronym in the acronym search results the available actions depend on whether the acronym is an industry standard or not. If the acronym is industry standard no further action is allowed. The user is provided only with the industry standard meaning for the acronym. If the acronym is not industry standard, the user may edit and update the meaning (or add to existing meaning) and save results under his/name as editor/contributor in database 11 record of acronyms.

A life science company makes use of literature for a wide variety of reasons. Among these are citation of literature to different regulatory agencies requiring different citation formats (e.g., EU verses US) or citation styles required by certain publications. When literature is used in several different contexts it can be extremely time consuming formatting the citation style for compliance for a particular use.

Referring to FIG. 6A there is shown a screenshot 59 of the display presented to the user by the GUI when the user accesses the citations styles menu. The screenshot shows a listing of the available citation styles 59a including standard (e.g., “Chicago style”) and custom style (“San Francisco style”), and citation creation portion 62, which provides boxes for selecting the format style (e.g., bold or italics) 62b, a format entry field 62c, bibliographic metadata 62a that is selected in the order that it will appear by selecting a metadata and pressing the “Insert” button. Once created a custom (or standard) citation for literature may be instantly created using metadata information associated with the literature.

FIG. 6B shows a flow diagram related to the software functionality described in connection with FIG. 6A. The flows shown are two: when using an existing citation style and creating a new citation style. When a new citation is added to the list citations, via the GUI the user clicks the add a new citation, the menu expands to show portion 62, select first piece of metadata that will appear first in the citation and choose font (e.g., bold or italic) and repeat this process until complete. Then save the citation under a new style, e.g., “Jim's style.”

When a literature citation is needed, e.g., for a written document, the user selects the Setting parts of the menu system presented by the GUI, a pop-up window appears showing all citation styles. Select the style that is desired, whereupon all citations in document are converted to the selected citation style.

As used herein a “web-based application” or “web app” should be taken to mean any program accessed over a network connection using HTTP, rather than existing within a device's memory. Web-based applications often run inside a web browser. However, web-based applications also may be client-based, where a small part of the program is downloaded to a user's desktop, but processing is done over the internet on an external server. Web-based applications are also known as web apps.

As used herein a “processor” should be taken to include one or a plurality of processors used to perform a task, such as executing SAS program code, processing data for a GUI, or reading or writing from memory and includes a general purpose computer.

As used herein a “user” should be taken to include an actual person using an application locally, or accessing the application remotely using a web browser using a computer located remotely. As used herein, a “computer” should be taken to include computers that have a hard disk drive for reading from and writing to a hard disk, a magnetic disk drive for reading from or writing to a removable magnetic disk, and an optical disk drive for reading from or writing to a removable optical disk such as a CD ROM, DVD, or other optical media. The hard disk drive, magnetic disk drive, and optical disk drive are connected to the system bus by a hard disk drive interface, a magnetic disk drive interface, and an optical drive interface, respectively. The drives and their associated computer-readable media provide nonvolatile (non-transitory) storage of computer readable instructions, data structures, programs, and other data for the computer system.

As used herein, a “database” should be taken to include a centralized or distributed database and includes a SQL database.

As used herein, the term “data” should be taken to include any information (excluding signals), contained in machine readable memory of a file system or used in a database or other structured data, or unstructured data representation in transitory or non-transitory memory.

As herein used, an “input device” should be taken to include a keyboard, mouse, pointer, touch pad, voice recognizer or other forms of input devices connected to a computer (with or without a display) for making modifications to, exploring, accessing, or generating electronic content that may be stored in memory.

As herein used, “display” is an LCD/LED type display or other types of display devices connected to the computer and capable of rendering and receiving input using a GUI or displaying other information generated by an application running on the computer executing machine executable code.

As used herein, “module” or “component” should be taken to include a software or source code file, or set of software instructions contained, or not contained in a source code file and existing on a non-transitory or transitory memory medium. A module may exist as a stand-alone program or represent a program called by another program. A “component” is a portion of a “module” in the sense that the module calls a component to perform a task or task(s) portion of the module.

As used herein “memory” should be taken to include a machine-readable medium, either transient computer-readable storage medium or non-transient computer-readable storage medium. In various embodiments, the volatile (transient) portion of memory may be implemented using any suitable memory technology, such as static random access memory (SRAM), synchronous dynamic RAM or any other type of memory. While the examples of memory as shown and described may refer to a single machine-readable medium, memory should be taken to include a single medium or multiple media (e.g., a centralized or distributed database, and/or associated caches and servers, and or a variety of storage media, such as the processor registers, memories, and the storage device) that store one or more sets of machine-executable code or data. The term “machine-readable medium” shall also be taken to include any medium that is capable of storing, encoding or carrying a set of instructions for execution by the machine and that cause the machine to perform any one or more of the methodologies of the present invention, or that is capable of storing, encoding or carrying data structures utilized by or associated with such a set of instructions. The term memory shall accordingly be taken to include tangible media, such as solid-state memories and optical and magnetic media.

As used herein “module” should be taken to include a unit of distinct functionality that can provide information to, and receive information from, other modules. Accordingly, described modules may be regarded as being communicatively coupled. Modules may also initiate communication with input or output devices, and can operate on a resource (e.g., a collection of information, such as a database). Modules may include hardware circuitry, optical components, single or multi-processor circuits, memory circuits, software program modules and objects, firmware, and combinations thereof, as appropriate for particular implementations of various embodiments. The term “module” can include an identifiable portion of code, data, or a computational object to achieve a particular function, operation, processing, or procedure.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in claims should not be construed to limit the invention to the specific embodiments disclosed in the specification.

Additional aspects of invention are described in the following CONCEPTS 1-20.

CONCEPT 1. A computer-implemented method, comprising:

providing a database for a life science company, the database comprising a corpus of scientific literature;

providing metadata for at least one of the literature on the company's database; and

sharing the literature with a person outside of the company, comprising the steps of:
calculate the number of copyrights available for the literature by accessing a copyrights portion of the metadata and if none available purchase an additional copyright, and
if the person is a health care professional perform the following steps before sharing the literature with the person:
access copyright information and a rights purchase history from the metadata to determine the copyright applying and value of a copy of the literature when shared with the person,
record the transaction and including indicating the name of the HCP and a value of the share with the health care professional (HCP) in the database.

CONCEPT 2. An apparatus comprising machine executable instructions or code stored in non-transitory memory that when executed by one or more processors performs the method of CONCEPT 1.

CONCEPT 3. The apparatus of CONCEPT 2, wherein the code when executed by the one or more processors the code further generates a plurality of graphical user interfaces (GUIs), comprising:
a first GUI for
   entering an address or name of the person to whom the literature will be shared, and
   indicating whether the person is a health care professional and if so, enabling a field for indicating the person's name and the number of copyrights used when the literature is shared with the person, a second GUI for
   displaying a purchase and sharing history for the literature, including a list of the persons to whom the literature was shared and a first cost field for a purchase of the literature and a second cost field for a sharing of the literature.

CONCEPT 4. The apparatus of CONCEPT 3, wherein the indicating whether the person is a health care professional comprises selecting a box to indicate that the Sunshine Act applies, whereupon the HCP name and number of copies fields are enabled.

CONCEPT 5. A system, comprising:

a database containing a life science company's corpus of scientific literature, the database comprising literature and metadata associated with the literature including purchase and copyright information;

one or more processors connected to a network and the company's database;

one or more input devices; and

machine executable code, residing in non-transitory memory accessible to the processor, that when executed by the processor performs the following tasks in response to a user's input received through the one or more input devices and indicating a desire to share the literature with a person outside of the company:
accessing the metadata on the database,
   calculating the number of copyrights available for the literature by accessing a copyrights portion of the metadata and if none available purchase an additional copyright, and
   if the person is a health care professional perform the following steps before sharing the literature with the person:
   access copyright information and a rights purchase history from the metadata to determine the copyright applying and value of a copy of the literature when shared with the person,
   record the transaction and including indicating the name of the HCP and a value of the share with the health care professional (HCP) in the database, and
   share the literature with the HCP.

CONCEPT 6. The system of CONCEPT 5, wherein the code when executed by the one or more processors the code further generates a plurality of graphical user interfaces (GUIs), comprising:
a first GUI for displaying the purchase history, number of copyrights, and whether the literature has been shared with anyone outside of the company
a second GUI for enabling entry of information including user sending, the person's address and a selectable box for whether the person is a HCP.

CONCEPT 7. A computer-implemented method, comprising:

providing a database for a life science company, the database comprising a corpus of scientific literature and a plurality of standard and company-specific acronyms used by the company;

displaying a result from a search of acronyms related to a user acronym entered as a search parameter;

if the search result returns no matching acronyms to the user acronym, perform steps comprising:
enter a definition for the user acronym, and
save the user acronym and definition in the database as a new company-specific acronym and link the user's name to the new company-specific acronym; and

if the search result returns one or more matching acronyms to the user acronym, perform steps comprising:

if the acronym is a standard acronym display the definition of the standard acronym, wherein the standard acronym cannot be modified by the user, and

if the acronym is a company-specific acronym, display the definition of the company-specific acronym and a prior one or more users who entered a comment, description or definition for the company-specific acronym, enable the user to add a new definition, comment or description, whereupon the new definition, comment or description is saved in the database under an existing record for the company-specific acronym and linked to the user's name.

CONCEPT 8. An apparatus comprising machine executable instructions or code stored in non-transitory memory that when executed by one or more processors performs the method of CONCEPT 7.

CONCEPT 9. A system, comprising:

a database containing a life science company's corpus of scientific literature, the database comprising literature and metadata associated with the literature including purchase and copyright information, and a plurality of company specific and standard acronyms, and definitions thereof;

one or more processors connected to a network and the company's database;

one or more input devices; and

machine executable code, residing in non-transitory memory accessible to the processor, that when executed by the processor performs the following tasks in response to a user's input received through the one or more input devices:

display a result from a search of acronyms related to a user acronym entered as a search parameter; and

if the search result returns no matching acronyms to the user acronym, perform steps comprising:
enter a definition for the user acronym, and
save the user acronym and definition in the database as a new company-specific acronym and link the user's name to the new company-specific acronym; and

if the search result returns one or more matching acronyms to the user acronym, perform steps comprising:

if the acronym is a standard acronym display the definition of the standard acronym, disable an editing mode preventing the user from modifying the standard acronym, and

if the acronym is a company-specific acronym, enable an editing mode allowing the user to modify the company-specific acronym, display the definition of the company-specific acronym and a prior one or more users who entered a comment, description or definition for the company-specific acronym, enable the user to add a new definition, comment or description, whereupon the new definition, comment or description is saved in the database under an existing record for the company-specific acronym and linked to the user's name.

CONCEPT 10. The system of CONCEPT 9, wherein the code when executed by the one or more processors the code further generates a plurality of graphical user interfaces (GUIs), comprising:
a first GUI for entering the search parameter and displaying results from the search, and
a second GUI for entering the new company-specific acronym name and definition, comment or description, enabled when there is no match in the search or an existing company specific acronym is selected by the user, and disabled when a standard acronym is selected or only a standard acronym is returned in the search results.

CONCEPT 11. A computer-implemented method for a user's acquisition of literature for a life science company having a database storing a corpus of scientific literature, the computer-implemented method comprising the steps of:

acquire the literature from a search of literature found on a remote database;

store purchase-related information for the acquisition under the user's account in the company's database;

store a copy of the literature in the company's database;

create metadata for the company's database, the metadata being generated and automatically linked to the literature, the metadata including
copyright costs,
one of a plurality of company programs associated with the literature,
bibliographic information, and
the user who acquired the literature.

CONCEPT 12. The method of CONCEPT 11, wherein the acquiring and storing steps include

select literature found in a search result for literature on the MEDLINE database,

request the literature from a content delivery service (CDS), and

receive purchase-related information about the literature from the CDS including a service cost and copyright cost,

wherein the metadata includes the purchase-related information.

CONCEPT 13. An apparatus comprising machine executable instructions or code stored in non-transitory memory that when executed by one or more processors performs the method of CONCEPT 11 or 12.

CONCEPT 14. A system for updating a corpus of scientific literature for a company, the system for managing the workflow comprising:

a processor;

one or more input devices;

a company database containing the corpus of scientific literature; and

machine executable code, residing in non-transitory memory accessible to the processor, that when executed by the processor performs the following tasks in response to a user's input received through the one or more input devices:

the machine executable code generating a plurality of graphical user interfaces (GUIs), comprising:
a first GUI for indicating which of a plurality of literature returned in a search of a remote database already exist on the company's database and literature subject to a copyright fee,
a second GUI for displaying a first set of contents of a metadata record linked to literature acquired from the remote database, the first set of contents of the metadata record being displayed as fields and listing one or more or any combination of
bibliographic information,
whether any of the authors of literature are from the company, one of a plurality of company programs associated with the literature, and company keywords
a third GUI for displaying a second set of contents of the metadata record linked to the literature acquired from the remote database, the second set of contents of the metadata record being displayed as fields and listing one or more or combination of
a purchase history for the literature including copyright purchases and number of copyrights available,
the user(s) purchasing or using copyrights, and
persons external to the company to which the literature was shared.

CONCEPT 15. A computer-implemented method, comprising:

providing a database for a life science company, the database comprising a corpus of scientific literature, metadata fields corresponding to bibliographic information that is linked to each of the literature, and a plurality of standard and custom citation styles for the literature;

displaying a list of existing citation styles; and

creating a new citation style, comprising steps of:
naming the new citation style
select a plurality of the metadata fields according to the order that bibliographic information about a literature will appear in the new citation style, and including defining a font type for selected ones of the metadata.

CONCEPT 16. An apparatus comprising machine executable instructions or code stored in non-transitory memory that when executed by one or more processors performs the method of CONCEPT 15.

CONCEPT 17. A system, comprising:

a database containing a life science company's corpus of scientific literature, the database comprising literature and metadata associated with the literature including purchase and copyright information, and a plurality of company specific and standard acronyms, and definitions thereof;

one or more processors connected to a network and the company's database;

one or more input devices; and

machine executable code, residing in non-transitory memory accessible to the processor, that when executed by the processor performs the following tasks in response to a user's input received through the one or more input devices:

display a result from a search of acronyms related to a user acronym entered as a search parameter; and

if the search result returns no matching acronyms to the user acronym, perform steps comprising:
enter a definition for the user acronym, and
save the user acronym and definition in the database as a new company-specific acronym and link the user's name to the new company-specific acronym; and

if the search result returns one or more matching acronyms to the user acronym, perform steps comprising:

if the acronym is a standard acronym display the definition of the standard acronym, disable an editing mode preventing the user from modifying the standard acronym, and

if the acronym is a company-specific acronym, enable an editing mode allowing the user to modify the company-specific acronym, display the definition of the company-specific acronym and a prior one or more users who entered a comment, description or definition for the company-specific acronym, enable the user to add a new definition, comment or description, whereupon the new definition, comment or description is saved in the database under an existing record for the company-specific acronym and linked to the user's name.

CONCEPT 18. An apparatus comprising machine executable instructions or code stored in non-transitory memory that when executed by one or more processors performs the method of CONCEPT 4.

CONCEPT 19. A system comprising:

a database containing a life science company's corpus of scientific literature, the database comprising literature and metadata associated with the literature including purchase and copyright information, and a plurality of company specific and standard acronyms, and definitions thereof;

one or more processors connected to the internet; and

machine executable instructions or code stored in non-transitory memory that when executed by one or more processors performs one or more of, or any combination of the methods of CONCEPTS 2, 4, 8, 13, 16 and 16, and

in response to user input searches and returns literature found according to a search parameter using the PUBMED search.

CONCEPT 20. An apparatus comprising machine executable instructions or code stored in non-transitory memory that when executed by one or more processors performs the method of CONCEPT 11 or 12, the method further including,

defining a library of literature within the life science company's corpus of scientific literature, the library being restricted to a user-define list of users, wherein only users who are members of the user-defined list have access rights to the library.

What is claimed is:

1. A computer-implemented method, comprising:
providing a database for a life science company, the database comprising a corpus of scientific literature;
providing metadata for at least one of the literature on the company's database; and
sharing the literature with a person outside of the company, comprising the steps of:
calculate the number of copyrights available for the literature by accessing a copyrights portion of the metadata and if there are an insufficient number of copyrights available, purchase additional copyrights, and
if the person is a health care professional perform the following steps before sharing the literature with the person:
access copyright information and rights purchase history from the metadata to determine the copyright applying and value of a copy of the literature when shared with the person,
record the transaction and including indicating the name of the health care professional (HCP) and a value of the share with the HCP in the database.

2. An apparatus comprising machine executable instructions or code stored in non-transitory memory that when executed by one or more processors performs the method of claim 1.

3. The apparatus of claim 2, wherein the code when executed by the one or more processors the code further generates a plurality of graphical user interfaces (GUIs), comprising:
a first GUI for
entering an email address or name of the person to whom the literature will be shared, and
indicating whether the person is a health care professional and if so, enabling a field for indicating the person's name and the number of copyrights used when the literature is shared with the person,
a second GUI for
displaying a purchase and sharing history for the literature, including a list of the persons to whom the literature was shared and a first cost field for a purchase of the literature and a second cost field for a sharing of the literature.

4. The apparatus of claim 3, wherein the indicating whether the person is a health care professional comprises selecting a box to indicate that the Sunshine Act applies, whereupon the HCP name and number of copies fields are enabled.

5. A system, comprising:
a database containing a life science company's corpus of scientific literature, the database comprising literature and metadata associated with the literature including purchase and copyright information;
one or more processors connected to a network and the company's database;
one or more input devices; and
machine executable code, residing in non-transitory memory accessible to the processor, that when executed by the processor performs the method of claim 1.

6. The system of claim 5, wherein the code when executed by the one or more processors the code further generates a plurality of graphical user interfaces (GUIs), comprising:
a first GUI for displaying the purchase history, number of copyrights, and whether the literature has been shared with anyone outside of the company
a second GUI for enabling entry of information including user sending, the person's address and a selectable box for whether the person is a health care professional (HCP).

7. A computer-implemented method, comprising:
providing a database for a life science company, the database comprising a corpus of scientific literature and a plurality of standard and company-specific acronyms used by the company;
displaying a result from a search of acronyms related to a user acronym entered as a search parameter;
if the search result returns no matching acronyms to the user acronym, perform steps comprising:
enter a definition for the user acronym, and
save the user acronym and definition in the database as a new company-specific acronym and link the user's name and when it was saved to the new company-specific acronym; and
if the search result returns one or more matching acronyms to the user acronym, perform steps comprising:
if the acronym is a standard acronym display the definition of the standard acronym, wherein the standard acronym cannot be modified by the user, and
if the acronym is a company-specific acronym, display the definition of the company-specific acronym and a prior one or more users who entered a comment, description or definition for the company-specific acronym, enable the user to add a new definition, comment or description, whereupon the new definition, comment or description is saved in the database under an existing record for the company-specific acronym and linked to the user's name.

8. An apparatus comprising machine executable instructions or code stored in non-transitory memory that when executed by one or more processors performs the method of claim 7.

9. A system, comprising:
a database containing a life science company's corpus of scientific literature, the database comprising literature and metadata associated with the literature including purchase and copyright information, and a plurality of company specific and standard acronyms, and definitions thereof;
one or more processors connected to a network and the company's database;
one or more input devices; and
machine executable code, residing in non-transitory memory accessible to the processor, that when executed by the processor performs the method of claim 7.

10. The system of claim 9, wherein the code when executed by the one or more processors the code further generates a plurality of graphical user interfaces (GUIs), comprising:
a first GUI for entering the search parameter and displaying results from the search, and
a second GUI for entering the new company-specific acronym name and definition, comment or description, enabled when there is no match in the search or an existing company specific acronym is selected by the user, and disabled when a standard acronym is selected or only a standard acronym is returned in the search results.

11. A computer-implemented method, the method comprising:
for a user's acquisition of literature for a life science company having a database storing a corpus of scientific literature, the computer-implemented method comprising the steps of:
acquire the literature from a search of literature full-text found on a remote database;
store purchase-related information for the acquisition under the user's account in the company's database;
store a copy of the literature in the company's database;
create metadata for the company's database, the metadata being generated and automatically linked to the literature, the metadata including
copyright costs,
one of a plurality of company programs associated with the literature,
bibliographic information, and
the user who acquired the literature.

12. The method of claim 11, wherein the acquiring and storing steps include
select literature found in a search result for literature on the MEDLINE database,
request the literature from a content delivery service (CDS), and
receive purchase-related information about the literature from the CDS including a service cost and copyright cost,
wherein the metadata includes the purchase-related information.

13. An apparatus comprising machine executable instructions or code stored in non-transitory memory that when executed by one or more processors performs the method of claim 12.

14. A system for updating a corpus of scientific literature for a company, the system for managing the workflow comprising:
a processor;
one or more input devices;
a company database containing the corpus of scientific literature; and
machine executable code, residing in non-transitory memory accessible to the processor, that when executed by the processor performs the method of claim 12.

15. The method of claim 11, further comprising
for the user's citation of literature for the life science company having the database storing a corpus of scientific literature, the computer-implemented method further comprising the steps of:
displaying a list of existing citation styles stored on the database; and
creating a new citation style, comprising steps of:
naming the new citation style
select a plurality of the metadata fields according to the order that bibliographic information about a literature will appear in the new citation style, and including defining a font type for selected ones of the metadata.

16. An apparatus comprising machine executable instructions or code stored in non-transitory memory that when executed by one or more processors performs the method of claim 15.
